(19) United States
(12) Patent Application Publication
(10) Pub. No.: US 2025/0334799 A1
(43) Pub. Date: Oct. 30, 2025

(54) MEASURING APPARATUS, MEASURING METHOD, AND STORAGE MEDIUM

(71) Applicant: CANON KABUSHIKI KAISHA, Tokyo (JP)

(72) Inventors: Toyokatsu FUJISAKI, Tochigi (JP); Kana KOBAYASHI, Tochigi (JP)

(21) Appl. No.: 19/262,288

(22) Filed: Jul. 8, 2025

Related U.S. Application Data
(63) Continuation of application No. PCT/JP2023/044943, filed on Dec. 14, 2023.

(30) Foreign Application Priority Data
Jan. 23, 2023 (JP) 2023-007810
Jan. 23, 2023 (JP) 2023-007849

(51) Int. Cl. G02B 27/00; G02B 27/01; H10F 30/225

(52) U.S. Cl. CPC G02B 27/0093; G02B 27/0172; H10F 30/225; G02B 2027/0138; G02B 2027/014

(57) ABSTRACT

A measuring apparatus for measuring a user's line-of-sight direction includes an illumination unit configured to emit light of a specific wavelength, a photodetector having a plurality of two-dimensionally arranged photoelectric converters, and configured to acquire light amount distribution information on reflected light of the light emitted by the illumination unit, one or more memories storing instructions, and one or more processors that, upon execution of the instructions, operate to calculate the line-of-sight direction based on the light amount distribution information. The light amount distribution information includes information on a time taken from when the illumination unit emits the light to when the photodetector detects the reflected light or a distance corresponding to the time, and information on an intensity of the reflected light.

### CROSS-REFERENCE TO RELATED APPLICATIONS

This application is a Continuation of International Patent Application No. PCT/JP2023/044943, filed on Dec. 14, 2023, which claims the benefit of Japanese Patent Applications Nos. 2023-007849 and 2023-007810, each filed on Jan. 23, 2023, both of which are hereby incorporated by reference herein in their entirety.

### BACKGROUND

### Field of the Technology

The present disclosure relates to a measuring apparatus, a measuring method, and a storage medium.

### Description of the Related Art

Conventionally, what is called line-of-sight (visual axis) detection devices (for example, eye cameras) configured to detect a position being observed by a user (viewer) are known. Japanese Patent Application Laid-Open No. 01-274736 discloses a method in which a parallel light beam from a light source is projected onto an anterior part of the user's eyeball and a line-of-sight direction is calculated based on a corneal reflection image produced by reflected light from the cornea and an imaging position of the pupil.

In the method disclosed in Japanese Patent Application Laid-Open No. 01-274736, in a case where the user is wearing glasses, it is impossible to distinguish reflected light from the cornea and reflected light from the glasses. As a result, reflected light from the glasses is sometimes mistaken for reflected light from the cornea in a case where the line-of-sight direction is calculated, which makes it impossible to perform highly accurate line-of-sight detection.

### SUMMARY

A measuring apparatus according to one aspect of the present disclosure for measuring a user's line-of-sight direction includes an illumination unit configured to emit light of a specific wavelength, a photodetector having a plurality of two-dimensionally arranged photoelectric converters, and configured to acquire light amount distribution information on reflected light of the light emitted by the illumination unit, one or more memories storing instructions, and one or more processors that, upon execution of the instructions, operate to calculate the line-of-sight direction based on the light amount distribution information. The light amount distribution information includes information on a time taken from when the illumination unit emits the light to when the photodetector detects the reflected light or a distance corresponding to the time, and information on an intensity of the reflected light. A measuring method corresponding to the above measuring apparatus and a storage medium storing a program that causes a computer to execute the above measuring apparatus also constitutes another aspect of the present disclosure.

A measuring apparatus according to another aspect of the present disclosure for measuring a user's line-of-sight direction includes an illumination unit configured to emit light of a specific wavelength, a photodetector having a plurality of two-dimensionally arranged photoelectric converters, and configured to acquire light amount distribution information on reflected light of the light emitted by the illumination unit, one or more memories storing instructions, and one or more processors that, upon execution of the instructions, operate to calculate the line-of-sight direction based on the light amount distribution information, calculate, based on the light amount distribution information, optical path length information from when the light is emitted by the illumination unit to when the light reaches the photodetector after reflection by a reflective surface, calculate a radius of curvature of the reflective surface based on the optical path length information, and calculate the line-of-sight direction based on the radius of curvature. A measuring method corresponding to the above measuring apparatus and a storage medium storing a program that causes a computer to execute the above measuring apparatus also constitute another aspect of the present disclosure.

Features of the present disclosure will become apparent from the following description of embodiments with reference to the attached drawings.

### BRIEF DESCRIPTION OF THE DRAWINGS

FIG. 1 is a block diagram of a measuring apparatus in a first embodiment and a second embodiment.

FIG. 2 is a configuration diagram of a pixel area of a photodetector in each embodiment.

FIG. 3 is an explanatory diagram of a drive pulse of the photodetector in each embodiment.

FIG. 4 is a flowchart illustrating the operation of the measuring apparatus in the first embodiment.

FIG. 5 is a flowchart illustrating operation of the measuring apparatus in the second embodiment.

FIG. 6 is a block diagram of a measuring apparatus in a third embodiment.

FIG. 7 is a flowchart illustrating the operation of the measuring apparatus in the third embodiment.

FIG. 8 is an explanatory diagram of the measuring apparatus in the first to third embodiments.

FIG. 9 is an explanatory diagram of intensity and time for reflected light from a cornea and other light in the first to third embodiments.

FIG. 10 is a block diagram of a measuring apparatus in a fourth embodiment and a fifth embodiment.

FIG. 11 is a flowchart illustrating operation of the measuring apparatus in the fourth embodiment.

FIG. 12 is a flowchart illustrating the operation of the measuring apparatus in the fifth embodiment.

FIG. 13 is a block diagram of a measuring apparatus in a sixth embodiment.

FIG. 14 is a flowchart illustrating operation of the measuring apparatus in the sixth embodiment.

FIG. 15 is an explanatory diagram of the measuring apparatus in the fourth to sixth embodiments.

FIG. 16 is an explanatory diagram of a method for calculating the radius of curvature in the fourth to sixth embodiments.

### DESCRIPTION OF THE EMBODIMENTS

In the following, the term “unit” may refer to a software context, a hardware context, or a combination of software and hardware contexts. In the software context, the term “unit” refers to a functionality, an application, a software module, a function, a routine, a set of instructions, or a program that can be executed by a programmable processor such as a microprocessor, a central processing unit (CPU), or a specially designed programmable device or controller. A memory contains instructions or programs that, when executed by the CPU, cause the CPU to perform operations corresponding to units or functions. In the hardware context, the term “unit” refers to a hardware element, a circuit, an assembly, a physical structure, a system, a module, or a subsystem. Depending on the specific embodiment, the term “unit” may include mechanical, optical, or electrical components, or any combination of them. The term “unit” may include active (e.g., transistors) or passive (e.g., capacitor) components. The term “unit” may include semiconductor devices having a substrate and other layers of materials having various concentrations of conductivity. It may include a CPU or a programmable processor that can execute a program stored in a memory to perform specified functions. The term “unit” may include logic elements (e.g., AND, OR) implemented by transistor circuits or any other switching circuits. In the combination of software and hardware contexts, the term “unit” or “circuit” refers to any combination of the software and hardware contexts as described above. In addition, the term “element,” “assembly,” “component,” or “device” may also refer to “circuit” with or without integration with packaging materials.

Referring now to the accompanying drawings, a detailed description will be given of embodiments according to the present disclosure. Corresponding elements in respective figures will be designated by the same reference numerals, and a duplicate description thereof will be omitted.

### First Embodiment

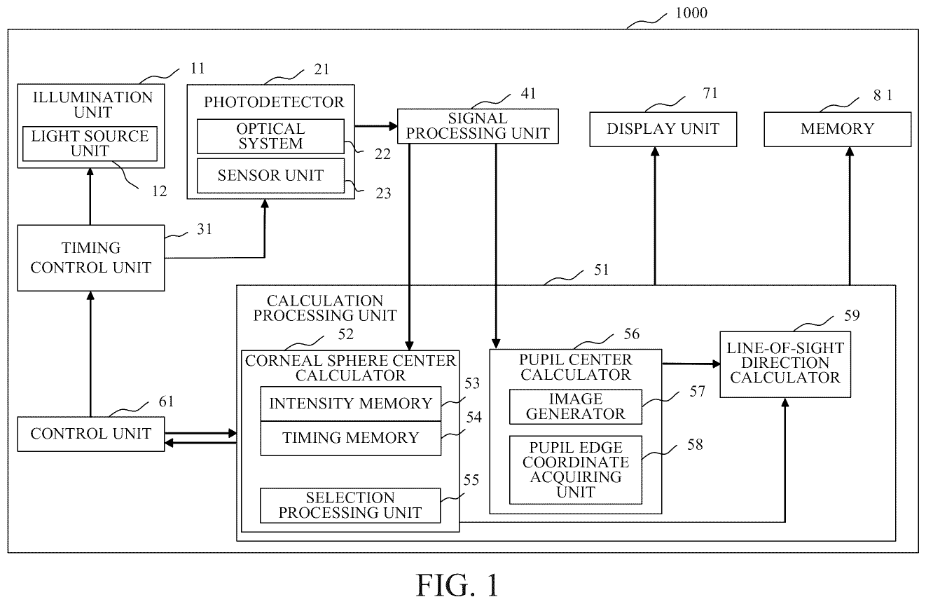

First, a measuring apparatus 1000 in a first embodiment of the present disclosure will be described below with reference to FIG. 1. FIG. 1 is a block diagram of the measuring apparatus 1000. The measuring apparatus 1000 includes an illumination unit 11, a photodetector 21, a timing control unit 31, a signal processing unit 41, a calculation processing unit 51, a control unit 61, a display unit 71, and a memory 81.

The illumination unit 11 includes a light source unit 12 in which a plurality of light sources are disposed. However, the present embodiment is not limited to this example but is also applicable to a light source unit in which only one light source is disposed. The light source unit 12 emits illumination light for illuminating an object (such as user's eye, eyeball, or glasses). The illumination light is light, the light amount of which changes with time, and is, for example, pulsed light or light modulated in a sine wave. The illumination light may be a pulsed laser beam of a specific wavelength or LED pulsed light. The object is irradiated by the illumination unit 11, and reflected light from the object is acquired by the photodetector 21. The illumination unit 11 may be fixed to a head-mounted display (HMD), an automobile (movable unit), or the like. The illumination unit 11 receives a light emission control signal transmitted from the timing control unit 31 and emits light in accordance with the light emission control signal. In the present embodiment, the light source unit 12 emits light of a specific wavelength with, for example, a width ranging from several picoseconds to several tens of nanoseconds.

The photodetector 21 receives light (reflected light) reflected by the object. The photodetector 21 acquires an optical image of the object formed through an optical system 22. A sensor unit 23 counts voltage pulses output from respective pixels of an image sensor (photoelectric conversion element) each time light is incident on the pixels (photon counting). The number of photon counts is output to the signal processing unit 41 together with a time at which each photon is counted. In the present embodiment, the photodetector 21 uses a time-of-flight (TOF) method. During one light receiving operation, the sensor unit 23 is exposed for a predetermined exposure time. The sensor unit 23 receives an exposure control signal transmitted from the timing control unit 31 and is exposed in accordance with the exposure control signal. During that time, a light source emits light one or more times with a predetermined pulse width, and the sensor unit 23 receives the light.

The timing control unit 31 controls the illumination unit 11 and the photodetector 21 so that the photodetector 21 receives the reflected light at the timing of irradiation by the illumination unit 11. The signal processing unit 41 performs processing suitable for subsequent processing on a signal output from the sensor unit 23. The processing performed by the signal processing unit 41 is, for example, signal processing such as noise removal but is not limited to this example. The signal processing unit 41 also maps an acquisition time based on a voltage pulse output from the photodetector 21 and a time at which a photon is counted.

The calculation processing unit 51 includes a corneal sphere center calculator 52, a pupil center calculator 56, and a line-of-sight direction calculator 59. The corneal sphere center calculator 52 includes an intensity memory 53, a timing memory 54, and a selection processing unit 55 and calculates a position coordinate at which the reflection position of the reflected light is imaged on a sensor surface of the sensor unit 23. The intensity memory 53 stores the intensity (the number of photons) of the reflected light among light amount distribution information on the reflected light. The timing memory 54 stores time or distance among the light amount distribution information. The selection processing unit 55 determines whether the reflected light is from a cornea based on a light reception signal output from the sensor unit 23 through the signal processing unit 41, a light reception time, an irradiation time from the timing control unit 31, and the number of photon counts or an intensity based on the number of counts. Then, the selection processing unit 55 selects only the reflected light from a cornea among the reflected light. The corneal sphere center calculator 52 calculates a cornea center by using the above-described result and outputs the calculation result to the line-of-sight direction calculator 59.

The pupil center calculator 56 includes an image generator 57 and a pupil edge coordinate acquiring unit 58. The image generator 57 acquires an image around a pupil. The pupil edge coordinate acquiring unit 58 acquires a pupil edge coordinate by using the image obtained by the image generator 57. The pupil center calculator 56 calculates a pupil center coordinate (pupil center position) based on the obtained image and the pupil edge coordinate and outputs the calculation result to the line-of-sight direction calculator 59. The line-of-sight direction calculator 59 calculates a line-of-sight direction based on the information obtained from the corneal sphere center calculator 52 and the information obtained from the pupil center calculator 56.

The control unit 61 comprehensively controls the operation of the measuring apparatus 1000. The control unit 61 includes a CPU and executes a computer program for controlling each component of the measuring apparatus 1000. The control unit 61 controls the calculation processing unit 51 in accordance with computer programs for controlling the illumination unit 11 and the photodetector 21. The control unit 61 may be implemented by one FPGA or the like together with at least one of the signal processing unit 41 or the calculation processing unit 51. The display unit 71 displays measurement results. The memory 81 accumulates image intensity data, time recording data, and line-of-sight direction data.

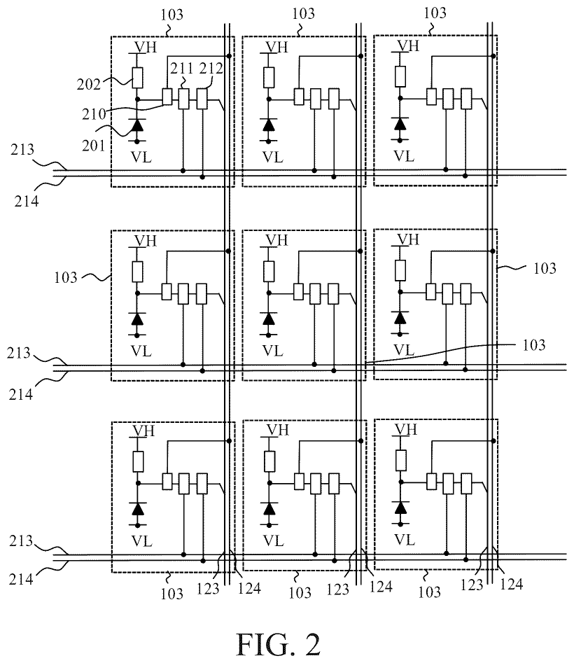

Next, a pixel area of the sensor unit 23 will be described below with reference to FIG. 2. FIG. 2 is a configuration diagram of the pixel area of the sensor unit 23. In the pixel area, SPAD pixels 103 are two-dimensionally arranged in a repeated pattern in X and Y directions. Each SPAD pixel 103 includes a photoelectric converter (avalanche diode) 201, a quench element 202, a control unit 210, a counter/memory 211, and a readout unit 212.

A potential based on a voltage VH higher than a voltage VL supplied to the anode is supplied to the cathode of the photoelectric converter 201. Potentials are supplied to the anode and cathode of the photoelectric converter 201 so that a reverse bias is applied to enable avalanche multiplication of photons incident on the photoelectric converter 201. In a case where photoelectric conversion is performed while such a reverse bias potential is supplied, the electric charge generated by incident light undergoes avalanche multiplication, which generates avalanche current.

In a case where the reverse bias potential is supplied and the potential difference between the anode and the cathode is larger than a breakdown voltage, the photoelectric converter (avalanche diode) 201 operates in a Geiger mode. An avalanche diode that uses the Geiger mode operation to detect weak signals at the single-photon level at high speed is referred to as a single photon avalanche diode (SPAD).

The control unit 210 determines whether to count signals output from the photoelectric converter 201. For example, the control unit 210 is a switch (gate circuit) provided between the photoelectric converter 201 and the counter/memory 211. The gate of the switch is connected to a pulse line 124, and the control unit 210 is switched on and off in accordance with a signal input to the pulse line 124. A signal based on a control signal from the timing control unit 31 is input to the pulse line 124. The gates of the switches are collectively controlled for all rows. Accordingly, start (light detection start) and end of light detection in all SPAD pixels 103 are collectively controlled.

The control unit 210 may be constituted by a logic circuit instead of a switch. For example, in a case where an AND circuit is provided as the logic circuit, a first input to the AND circuit is an output from the photoelectric converter 201, and a second input thereto is a signal from the pulse line 124, it is possible to switch whether to count signals output from the photoelectric converter 201. The control unit 210 does not necessarily need to be provided between the photoelectric converter 201 and the counter/memory 211 but may be a circuit that inputs a signal for switching counter operation in the counter/memory 211.

In accordance with a control signal from a control line 213, the counter/memory 211 counts the number of photons entering the photoelectric converter 201 and holds the number as digital data. The readout unit 212 is connected to the counter/memory 211 and a reading signal line 123. A control pulse is supplied to the readout unit 212 from a vertical scanning circuit unit through a control line 214, and whether to output the count value in the counter/memory 211 to the reading signal line 123 is switched. The readout unit 212 includes, for example, a buffer circuit for outputting signals.

The reading signal line 123 may be a signal line for outputting from the photodetector 21 to the calculation processing unit 51, or a signal line for outputting to the signal processing unit 41. Moreover, a horizontal scanning circuit unit and the vertical scanning circuit unit may be provided on a substrate on which a SPAD array is provided may be provided on a substrate different from the substrate on which the SPAD array is provided.

However, the present embodiment is not limited to the photodetector 21 including the SPAD pixels 103, but is also applicable to a photodetector including a photoelectric converter other than SPAD.

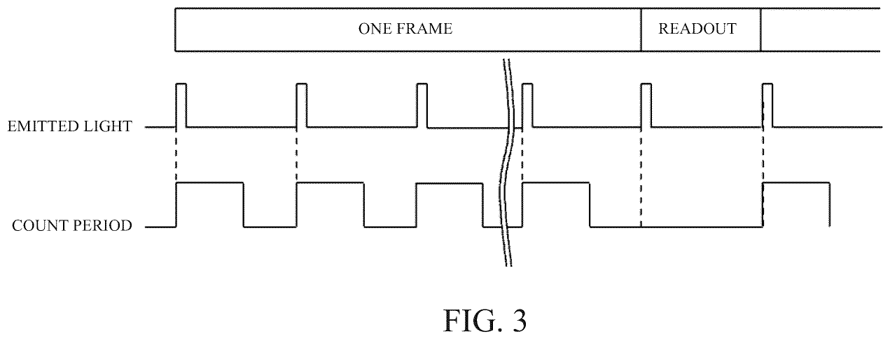

Next, a timing (emission timing) at which pulsed light from the illumination unit 11 is emitted, and timings at which the pulsed light is emitted to the object (emission is started), the reflected light reaches the photodetector 21, and light detection (photon counting) is performed will be described below with reference to FIG. 3. FIG. 3 is an explanatory diagram of a drive pulse of the photodetector 21. In the photodetector 21, the plurality of SPAD pixels 103 are arranged in an array, and light detection timings of a plurality of SPADs disposed on each row are collectively controlled for all pixels. In other words, the timing of light emission and the timing of count period, which are illustrated in FIG. 3, are the same in all SPAD pixels in one frame.

The photodetector 21 is a SPAD array in which the SPAD pixels 103 are two-dimensionally arranged. Accordingly, with the timing chart as described above, it is possible to acquire, for each frame, a data set (light amount distribution information) including light amount distribution information (x, y) on an XY plane and time information (t) of a time at which the light amount distribution information is acquired. Thus, it is possible to acquire information (light amount distribution information) related to x, y, and t.

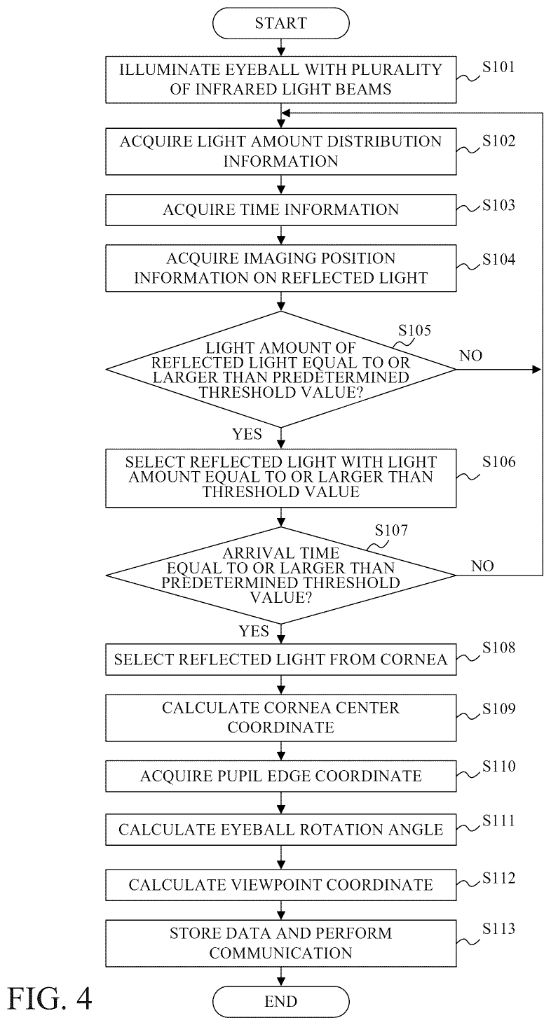

Next, the operation of the measuring apparatus 1000 (measuring method) in the present embodiment will be described below with reference to FIG. 4. FIG. 4 is a flowchart illustrating the operation of the measuring apparatus 1000. Each step in FIG. 4 is mainly executed by components of the measuring apparatus 1000 based on commands from the control unit 61.

First in step S101, the illumination unit 11 illuminates an object such as a user's eye (eyeball) with light of a specific wavelength (infrared light), which is emitted from the light source unit 12 including a plurality of light sources. The light of a specific wavelength is not limited to infrared light but may be light of any other wavelength. Next in step S102, the photodetector 21 detects reflected light from the object and acquires light amount distribution information. The light amount distribution information refers to the number of photon counts incident on each pixel or an image luminance value calculated from the number of counts, and the position coordinate (pixel coordinate) of the pixel.

Next in step S103, the calculation processing unit 51 acquires the time of light emission from the light source unit 12 and the time of light reception at the sensor unit 23, and acquires time information from in a case where light is emitted from the light source unit 12 to in a case where the light reaches the sensor unit 23 after reflection by the object. The acquisition in steps S102 and S103 may be simultaneously performed by the sensor unit 23. Next in step S104, the calculation processing unit 51 calculates (acquires) a reflective surface position coordinate of the reflected light (imaging position information on the reflected light) based on the information obtained in step S102 and the information obtained in step S103. Next in step S105, it is determined whether the light amount of the reflected light (intensity of the reflected light; for example, the number of photons having arrived at the photodetector 21) among the light amount distribution information is equal to or larger than a predetermined threshold value (intensity threshold value or second threshold value). In a case where the light amount (intensity) of the reflected light is smaller than the second threshold value, the calculation processing unit 51 determines that the reflected light is unnecessary light (second reflected light) that is different from reflected light from a cornea (first reflected light), and returns to step S102 to acquire different light amount distribution information. When having determined that the light amount (intensity) of the reflected light is equal to or larger than the second threshold value, the calculation processing unit 51 proceeds to step S106. In step S106, the calculation processing unit 51 selects reflected light with a light amount (intensity) equal to or larger than the second threshold value.

Next in step S107, the calculation processing unit 51 refers to the time information (arrival time) acquired in step S103 and determines whether the time information is equal to or larger than a predetermined threshold value (time threshold value or first threshold value). The time information is information on a time taken from when the illumination unit 11 emits light to when the photodetector 21 detects reflected light or to a distance corresponding to the time. In a case where the time or the distance is smaller than the first threshold value, the calculation processing unit 51 determines that the reflected light is unnecessary light (second reflected light) that is different from reflected light from a cornea (first reflected light), and returns to step S102 to acquire different light amount distribution information. In a case where the time or the distance is equal to or larger than the first threshold value, the calculation processing unit 51 proceeds to step S108.

In step S108, the calculation processing unit 51 (selection processing unit 55) selects reflected light, the time information on which is determined to be equal to or larger than the first threshold value as reflected light from a cornea. Next in step S109, the calculation processing unit 51 (corneal sphere center calculator 52) selects a plurality of beams of reflected light from the cornea and calculates a cornea center coordinate. Next in step S110, the calculation processing unit 51 (pupil center calculator 56) acquires a pupil image and a pupil edge coordinate. The pupil image may be acquired by the sensor unit 23 or may be acquired by another sensor through image pickup. The image pickup may be performed by a sensor with a TOF function, and the pupil image may be acquired by using what is called a CMOS camera with no TOF function.

Next in step S111, the calculation processing unit 51 calculates a pupil center coordinate. Specifically, the calculation processing unit 51 calculates the rotation angle of the eyeball based on the cornea center coordinate acquired in step S109 and the pupil edge coordinate acquired in step S110. Next in step S112, the calculation processing unit 51 calculates a viewpoint coordinate and detects the line-of-sight direction of the user. Next in step S113, the calculation processing unit 51 stores data and performs communication with the control unit 61, the display unit 71, the memory 81, or the like.

### Second Embodiment

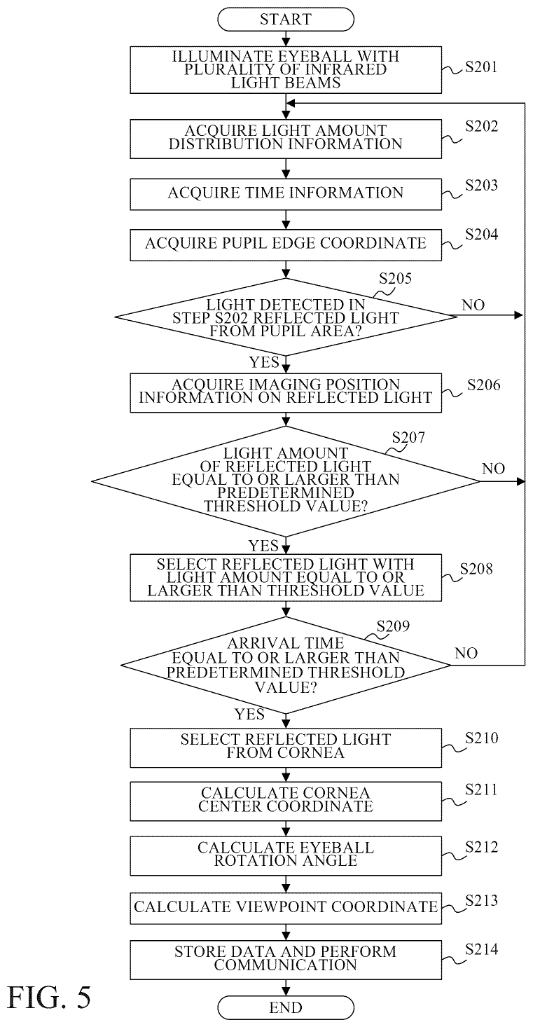

Next, operation of the measuring apparatus 1000 (measuring method) in a second embodiment of the present disclosure will be described below with reference to FIG. 5. FIG. 5 is a flowchart illustrating the operation of the measuring apparatus 1000 in the present embodiment. Each step in FIG. 5 is mainly executed by components of the measuring apparatus 1000 based on commands from the control unit 61. The basic configuration of the measuring apparatus 1000 of the present embodiment is the same as the measuring apparatus 1000 of the first embodiment described above with reference to FIG. 1, and thus a description thereof will be omitted.

First in step S201, the illumination unit 11 illuminates an object such as a user's eye (eyeball) with light of a specific wavelength (infrared light), which is emitted from the light source unit 12 including a plurality of light sources. Next in step S202, the photodetector 21 detects reflected light from the object and acquires light amount distribution information including luminance information (intensity information) of the reflected light. Next in step S203, the calculation processing unit 51 acquires the time of light emission from the light source unit 12 and the time of light reception at the sensor unit 23, and acquires time information from when light is emitted from the light source unit 12 to when the light reaches the sensor unit 23 after reflection by the object. Next in step S204, the calculation processing unit 51 (pupil center calculator 56) acquires a pupil edge coordinate and a pupil center coordinate by using a pupil image acquired by the photodetector 21. Next in step S205, the calculation processing unit 51 determines whether the reflected light detected in step S202 is reflected light from a pupil area based on luminance information on the image (reflected light). In a case where it is determined that the detected reflected light is not reflected light from a pupil area, the process returns to step S202 to acquire different light amount distribution information. In a case where it is determined that the detected reflected light is reflected light from a pupil area, the process proceeds to step S206.

In step S206, the calculation processing unit 51 calculates (acquires) a reflective surface position coordinate of the reflected light (imaging position information on the reflected light) based on the information obtained in step S202 and the information obtained in step S203. Next in step S207, it is determined whether the light amount of the reflected light (intensity of the reflected light) among the light amount distribution information is equal to or larger than a predetermined threshold value (intensity threshold value or second threshold value). In a case where the light amount (intensity) of the reflected light is smaller than the second threshold value, the calculation processing unit 51 determines that the reflected light is unnecessary light (second reflected light), and returns to step S202 to acquire different light amount distribution information. When having determined that the light amount (intensity) of the reflected light is equal to or larger than the second threshold value, the calculation processing unit 51 proceeds to step S208. In step S208, the calculation processing unit 51 selects reflected light with a light amount (intensity) equal to or larger than the second threshold value.

Next in step S209, the calculation processing unit 51 refers to the time information (arrival time) acquired in step S203 and determines whether the time information is equal to or larger than a predetermined threshold value (time threshold value or first threshold value). In a case where the time information is smaller than the first threshold value, the calculation processing unit 51 determines that the reflected light is unnecessary light (second reflected light), and returns to step S202 to acquire different light amount distribution information. In a case where the time information is equal to or larger than the first threshold value, the calculation processing unit 51 proceeds to step S210.

In step S210, the calculation processing unit 51 (selection processing unit 55) selects reflected light the time information on which is determined to be equal to or larger than the first threshold value as reflected light from a cornea. Next in step S211, the calculation processing unit 51 (corneal sphere center calculator 52) selects a plurality of beams of reflected light from the cornea and calculates a cornea center coordinate.

Next in step S212, the calculation processing unit 51 calculates a pupil center coordinate. Specifically, the calculation processing unit 51 calculates the rotation angle of the eyeball based on the cornea center coordinate acquired in step S211 and the pupil edge coordinate acquired in step S204. Next in step S213, the calculation processing unit 51 calculates a viewpoint coordinate and detects the line-of-sight direction of the user. Next in step 214, the calculation processing unit 51 stores data and performs communication with the control unit 61, the display unit 71, the memory 81, or the like.

### Third Embodiment

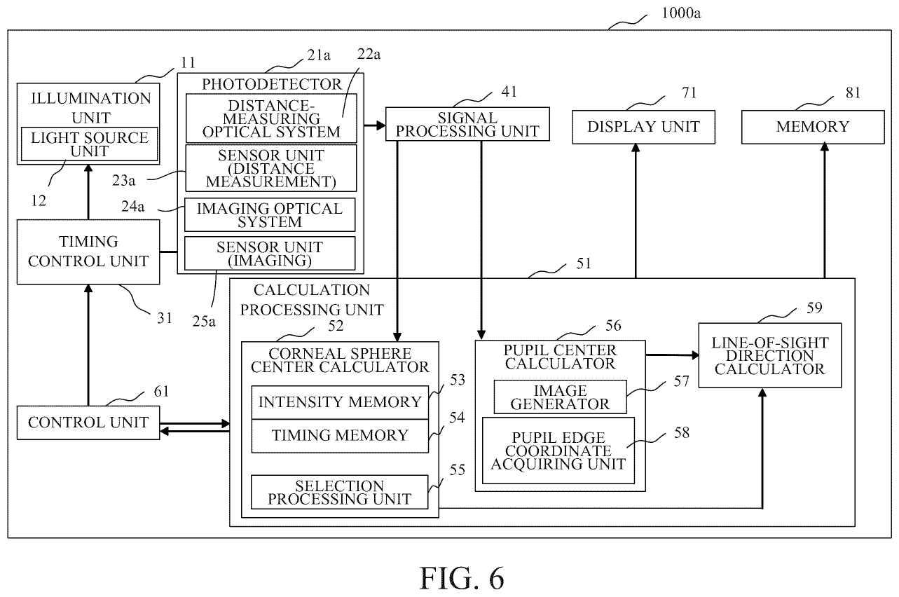

Next, a measuring apparatus 1000a in a third embodiment of the present disclosure will be described below with reference to FIG. 6. FIG. 6 is a block diagram of the measuring apparatus 1000a. The measuring apparatus 1000a is different from the measuring apparatus 1000 in that a photodetector 21a is provided in place of the photodetector 21. The other configuration of the measuring apparatus 1000a is the same as that of the measuring apparatus 1000, and thus a description thereof will be omitted.

The photodetector 21a receives light (reflected light) reflected by an object. The photodetector 21a includes a distance-measuring optical system 22a, a sensor unit (distance measurement sensor unit) 23a, an imaging optical system 24a, and a sensor unit (image pickup sensor unit) 25a. The photodetector 21a condenses the reflected light through the distance-measuring optical system 22a to image the light onto the sensor unit 23a, and the photodetector 21a outputs a signal to the signal processing unit 41 in accordance with light amount distribution information obtained by the sensor unit 23a, in other words, the intensity and acquisition time (time information) of the reflected light. In the present embodiment, the distance-measuring optical system 22a and the sensor unit 23a use a time-of-flight (TOF) method.

The imaging optical system 24a and the sensor unit 25a pick up an image of a user's eyeball (eye) to acquire an image for calculating a pupil center. Light amount distribution information obtained by the sensor unit 25a is output to the signal processing unit 41. The sensor unit 25a is an image sensor (photoelectric conversion element) such as a CCD sensor or a CMOS sensor.

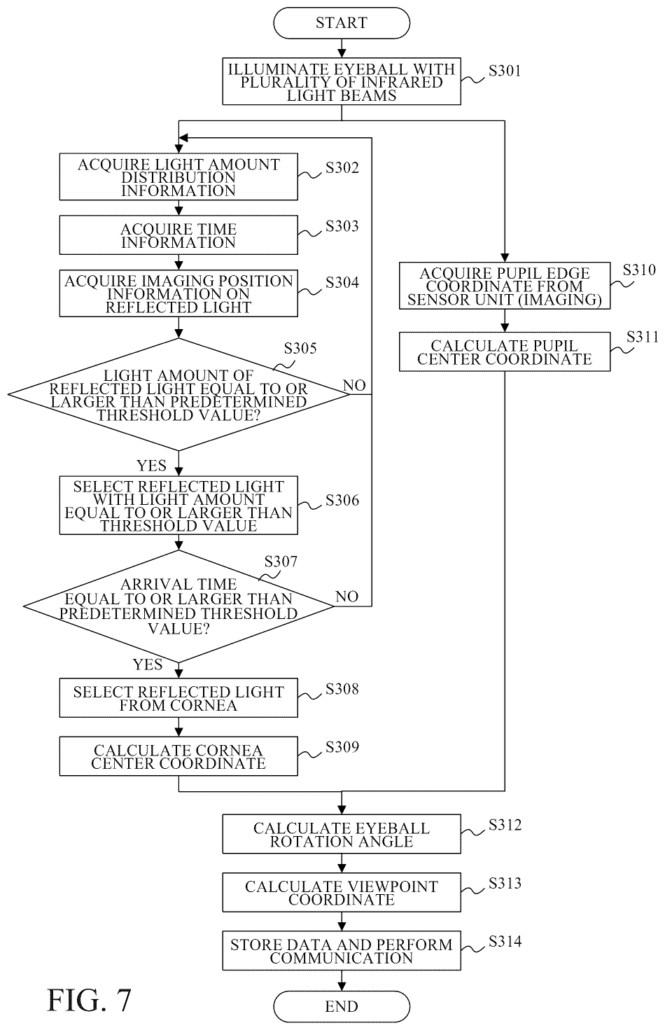

Next, operation of the measuring apparatus 1000a (measuring method) in the present embodiment will be described below with reference to FIG. 7. FIG. 7 is a flowchart illustrating the operation of the measuring apparatus 1000a. Each step in FIG. 7 is mainly executed by components of the measuring apparatus 1000a based on commands from the control unit 61.

First in step S301, the illumination unit 11 illuminates an object such as a user's eye (eyeball) with light of a specific wavelength (infrared light), which is emitted from the light source unit 12 including a plurality of light sources. Next in step S302, the photodetector 21a detects reflected light from the object by using the sensor unit 23a and acquires light amount distribution information including luminance information (intensity information) of the reflected light. Next in step S303, the calculation processing unit 51 acquires the time of light emission from the light source unit 12 and the time of light reception at the sensor unit 23a, and acquires time information from when light is emitted from the light source unit 12 to when the light reaches the sensor unit 23a after reflection by the object.

Next in step S304, the calculation processing unit 51 calculates (acquires) a reflective surface position coordinate of the reflected light (imaging position information on the reflected light) based on the information obtained in step S302 and the information obtained in step S303. Next in step S305, it is determined whether the light amount of the reflected light (intensity of the reflected light) among the light amount distribution information is equal to or larger than a predetermined threshold value (intensity threshold value or second threshold value). In a case where the light amount (intensity) of the reflected light is smaller than the second threshold value, the calculation processing unit 51 determines that the reflected light is unnecessary light (second reflected light), and returns to step S302 to acquire different light amount distribution information. When having determined that the light amount (intensity) of the reflected light is equal to or larger than the second threshold value, the calculation processing unit 51 proceeds to step S306. In step S306, the calculation processing unit 51 selects reflected light with a light amount (intensity) equal to or larger than the second threshold value.

Next in step S307, the calculation processing unit 51 refers to the time information (arrival time) acquired in step S303 and determines whether the time information is equal to or larger than a predetermined threshold value (time threshold value or first threshold value). In a case where the time or the distance is smaller than the first threshold value, the calculation processing unit 51 determines that the reflected light is unnecessary light (second reflected light), and returns to step S302 to acquire different light amount distribution information. In a case where the time or the distance is equal to or larger than the first threshold value, the calculation processing unit 51 proceeds to step S308.

In step S308, the calculation processing unit 51 (selection processing unit 55) selects reflected light the time information on which is determined to be equal to or larger than the first threshold value as reflected light from a cornea. Next in step S309, the calculation processing unit 51 (corneal sphere center calculator 52) selects a plurality of beams of reflected light from the cornea and calculates a cornea center coordinate.

In step S310, the calculation processing unit 51 (pupil center calculator 56) acquires a pupil image from the sensor unit 25a and acquires a pupil edge coordinate. Next in step S311, the calculation processing unit 51 calculates a pupil center coordinate based on the pupil edge coordinate.

Next in step S312, the calculation processing unit 51 calculates a pupil center coordinate. Specifically, the calculation processing unit 51 calculates the rotation angle of the eyeball based on the cornea center coordinate acquired in step S309 and the pupil center coordinate acquired in step S311. Next in step S313, the calculation processing unit 51 calculates a viewpoint coordinate and detects the line-of-sight direction of the user. Next in step 314, the calculation processing unit 51 stores data and performs communication with the control unit 61, the display unit 71, the memory 81, or the like. The processing steps S302 to S309 and the processing steps S310 to S311 may be simultaneously performed.

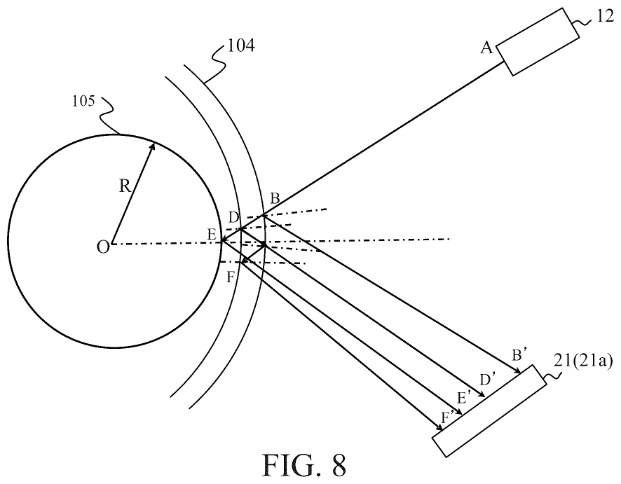

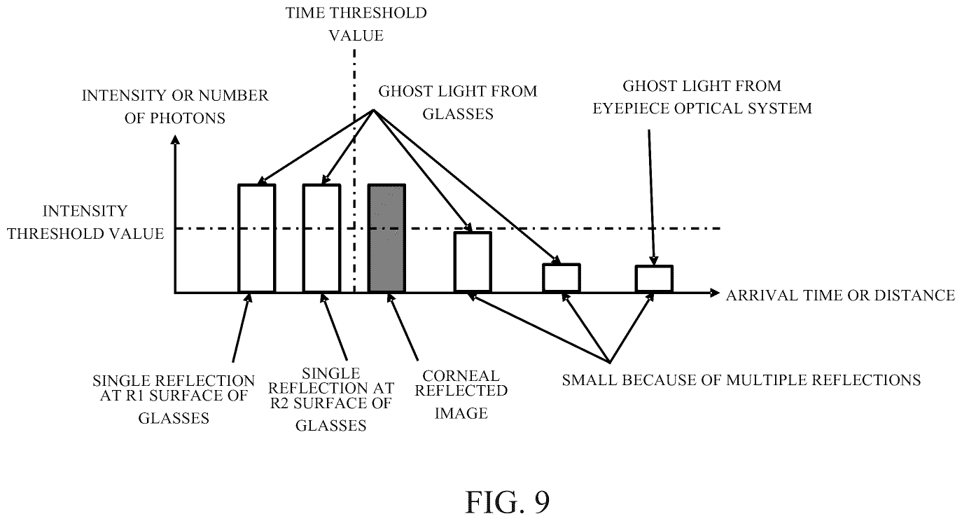

Next, processing of separating reflected light from a cornea (first reflected light) and the other unnecessary light (second reflected light) in the first to third embodiments will be described below with reference to FIGS. 8 and 9. FIG. 8 is an explanatory diagram of the measuring apparatus in the first to third embodiments. FIG. 9 is an explanatory diagram of intensity and time for the reflected light (first reflected light) from the cornea and the other reflected light (second reflected light).

In FIG. 8, O denotes the center of the eyeball, and R denotes the radius of the cornea, which is 7 to 8 mm approximately for a typical human eye. Pulsed light emitted from the light source unit 12 is reflected by an eyeball part 105 and a glass part 104 and reaches the photodetector 21 (21a). At the glass part 104, there exist light reflected once by a surface B on the light source unit 12 side and a surface D on the eyeball side, and light reflected a plurality of times inside and arriving at the photodetector 21 (21a).

Among these lights, light reflected by a cornea surface E of the eyeball part 105 reaches the photodetector 21 (21a) later than light reflected by the surface B of the glass part 104 on the light source side and the surface D thereof on the eyeball side because the optical path length is longer. Light reflected a plurality of times and finally reflected by a surface F of the glass part 104 before arriving at the photodetector 21 arrives simultaneously with or later than light reflected by the cornea surface E of the eyeball part 105. Light reflected a plurality of times by the glass part 104 results in a smaller number of photons arriving at the photodetector 21 (21a) than light reflected by the surface E of the eyeball part 105, which leads to weaker image intensity.

In general, reflection by an eyeball is 2 to 3% approximately, and the reflectance of typical glasses with glass and plastic lenses is often equal to or smaller than 2% even in the near-infrared region. Thus, as the number of reflections increases, the number of photons arriving at the photodetector 21 (21a) decreases and the image intensity becomes weaker. Although the glass part 104 is illustrated as a cause of unnecessary light reflection in FIG. 8, the causes of unnecessary light are not limited to this example. Other causes of unnecessary light include, for example, a case where an eyepiece of an electronic viewfinder or a liquid crystal panel is disposed in front of the eyeball part 105.

FIG. 9 is a graph illustrating a case where the photodetector 21 (21a) detects unnecessary light (second reflected light) reflected by the glass part 104 or an eyepiece lens part and normal light (first reflected light) from a cornea for line-of-sight detection. In FIG. 9, the vertical axis represents intensity or the number of photons, and the horizontal axis represents a time or distance until light from the light source unit 12 reaches the photodetector 21 (21a). As illustrated in FIG. 9, threshold value (intensity threshold value or second threshold value) for the intensity (luminance) of reflected light and a threshold value (time threshold value or first threshold value) for the arrival time are set. This makes it possible to determine whether light detected by the photodetector 21 (21a) is reflected light from a cornea (first reflected light) or reflected light (second reflected light) that is unnecessary.

### Fourth Embodiment

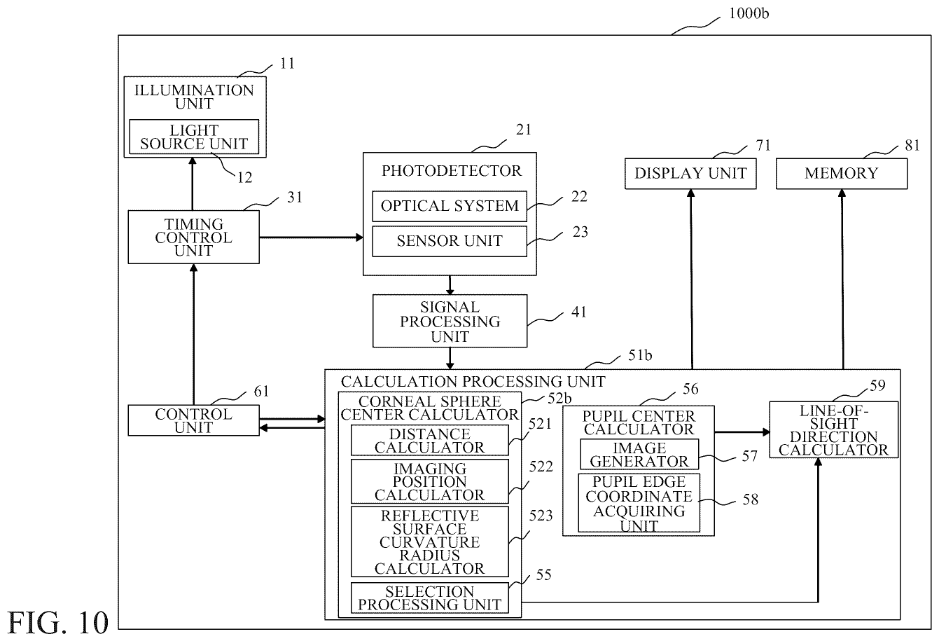

First, a measuring apparatus 1000b in a fourth embodiment of the present disclosure will be described below with reference to FIG. 10. FIG. 10 is a block diagram of the measuring apparatus 1000b. The measuring apparatus 1000b includes the illumination unit 11, the photodetector 21, the timing control unit 31, the signal processing unit 41, a calculation processing unit 51b, the control unit 61, the display unit 71, and the memory 81.

The illumination unit 11 includes the light source unit 12 in which three or more light sources are disposed. The light source unit 12 emits illumination light for illuminating an object (user's eye, eyeball, or glasses). The illumination light is light the light amount of which changes with time, and is, for example, pulsed light or light modulated in a sine wave. The illumination light may be a pulsed laser beam of a specific wavelength or LED pulsed light. The object is irradiated by the illumination unit 11, and reflected light from the object is acquired by the photodetector 21. The illumination unit 11 may be fixed to a head-mounted display (HMD), an automobile (movable unit), or the like. The illumination unit 11 receives a light emission control signal transmitted from the timing control unit 31 and emits light in accordance with the light emission control signal. In the present embodiment, the light source unit 12 emits light of a specific wavelength with, for example, a width ranging from several picoseconds to several tens of nanoseconds.

The photodetector 21 receives light (reflected light) reflected by the object. The photodetector 21 acquires an optical image of the object formed through the optical system (distance measurement optical system) 22. The sensor unit 23 counts voltage pulses output from respective pixels of an image sensor (photoelectric conversion element) each time light is incident on the pixels (photon counting). The number of photon counts is output to the signal processing unit 41 together with a time at which each photon is counted. In the present embodiment, the photodetector 21 uses a time of flight (TOF) method. During one light receiving operation, the sensor unit 23 is exposed for a predetermined exposure time. The sensor unit 23 receives an exposure control signal transmitted from the timing control unit 31 and is exposed in accordance with the exposure control signal. During that time, the light source emits light one or more times with a predetermined pulse width, and the sensor unit 23 receives the light.

The timing control unit 31 controls the illumination unit 11 and the photodetector 21 so that the photodetector 21 receives the reflected light at the timing of irradiation by the illumination unit 11. The signal processing unit 41 performs processing suitable for subsequent processing on a signal output from the sensor unit 23. The processing performed by the signal processing unit 41 is, for example, signal processing such as noise removal but is not limited to this example. The signal processing unit 41 also maps an acquisition time based on a voltage pulse output from the photodetector 21 and a time at which a photon is counted.

The calculation processing unit 51b includes a corneal sphere center calculator 52b, the pupil center calculator 56, and the line-of-sight direction calculator 59. The corneal sphere center calculator 52b includes a distance calculator 521, an imaging position calculator 522, a reflective surface curvature radius calculator 523, and the selection processing unit 55. The distance calculator 521 calculates an optical path length (light beam flight distance) of received light for each pixel based on a light reception signal output from the sensor unit 23 through the signal processing unit 41, a light reception time, and an irradiation time from the timing control unit 31. The imaging position calculator 522 calculates a position coordinate at which the reflection position is imaged on the sensor surface of the sensor unit 23. The reflective surface curvature radius calculator 523 reads the optical path length of each pixel, position information on the light source unit 12 and the sensor unit 23, which is stored in a program memory in advance, and a reflective surface curvature radius calculation program, and calculates a reflective surface curvature radius based on designated reflected light. The selection processing unit 55 determines, based on the calculated reflective surface curvature radius, whether the reflected light is reflected light from a cornea (first reflected light) or reflected light (second reflected light) that is unnecessary, and selects only the reflected light from a cornea (first reflected light).

The corneal sphere center calculator 52b calculates a cornea center by using the above-described result and outputs the calculation result to the line-of-sight direction calculator 59. The pupil center calculator 56 includes the image generator 57 and the pupil edge coordinate acquiring unit 58. The image generator 57 acquires an image around the pupil. The pupil edge coordinate acquiring unit 58 acquires a pupil edge coordinate by using the image obtained by the image generator 57. The pupil center calculator 56 calculates a pupil center coordinate based on the obtained image and the pupil edge coordinate and outputs the calculation result to the line-of-sight direction calculator 59. The line-of-sight direction calculator 59 calculates a line-of-sight direction based on the information obtained from the corneal sphere center calculator 52b and the information obtained from the pupil center calculator 56.

The control unit 61 collectively controls the operation of the measuring apparatus 1000b. The control unit 61 includes a CPU and executes a computer program for controlling each component of the measuring apparatus 1000b. The control unit 61 controls the calculation processing unit 51 in accordance with computer programs for controlling the illumination unit 11 and the photodetector 21. The control unit 61 may be implemented by one FPGA or the like together with at least one of the signal processing unit 41 or the calculation processing unit 51. The display unit 71 displays measurement results. The memory 81 accumulates distance image data and line-of-sight direction data.

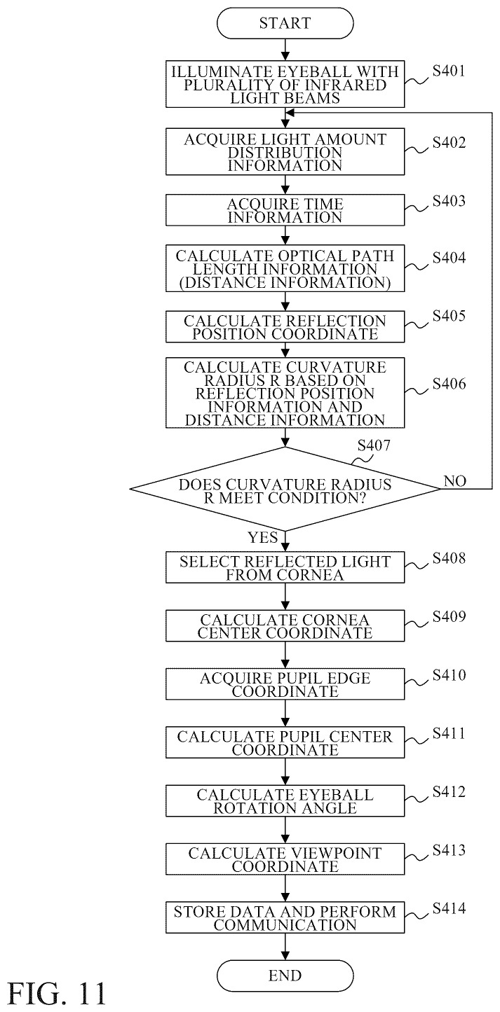

Next, the operation of the measuring apparatus 1000b (measuring method) in the present embodiment will be described below with reference to FIG. 11. FIG. 11 is a flowchart illustrating the operation of the measuring apparatus 1000b. Each step in FIG. 11 is mainly executed by components of the measuring apparatus 1000b based on commands from the control unit 61.

First in step S401, the illumination unit 11 illuminates an object such as a user's eye (eyeball) with light of a specific wavelength (infrared light), which is emitted from the light source unit 12 including a plurality of light sources. The light of a specific wavelength is not limited to infrared light but may be light of any other wavelength. Next in step S402, the photodetector 21 detects reflected light from the object and acquires light amount distribution information. The light amount distribution information refers to the number of photon counts incident on each pixel or an image luminance value calculated from the number of counts, and the position coordinate (pixel coordinate) of the pixel. Next in step S403, the calculation processing unit 51 acquires the time of light emission from the light source unit 12 and the time of light reception at the sensor unit 23, and acquires time information from when light is emitted from the light source unit 12 to when the light reaches the sensor unit 23 after reflection by the object. The acquisition in steps S402 and S403 may be simultaneously performed by the sensor unit 23.

Next in step S404, the calculation processing unit 51 (distance calculator 521) calculates optical path length information (distance information) based on the light amount distribution information acquired in step S402 and the time information acquired in step S403. The optical path length information is information on an optical path length from when light is emitted from the light source unit 12 of the illumination unit 11 to when the light reaches the sensor unit 23 of the photodetector 21 after reflection by the object (reflective surface). Next in step S405, the calculation processing unit 51 (imaging position calculator 522) calculates a reflective surface position coordinate (reflection position information) of the reflected light based on the optical path length information acquired in step S404. Next in step S406, the calculation processing unit 51 (reflective surface curvature radius calculator 523) calculates a radius R of curvature of the object (reflective surface) based on the reflection position coordinate (reflection position information) acquired in step S405 and the distance information acquired in step S404.

Next in step S407, the calculation processing unit 51 determines whether the radius R of curvature acquired in step S406 satisfies a predetermined condition, in other words, whether the radius R of curvature is within a predetermined range. In a case where it is determined that the radius R of curvature does not satisfy the predetermined condition, the process returns to step S402 to acquire different light amount distribution information. In a case where it is determined that the radius R of curvature satisfies the predetermined condition, the process proceeds to step 108.

In step S408, the calculation processing unit 51 (selection processing unit 55) selects reflected light for which the radius R of curvature is determined to be within the predetermined range as reflected light from a cornea. Next in step S409, the calculation processing unit 51 (corneal sphere center calculator 52b) selects a plurality of beams of reflected light from the cornea and calculates a cornea center coordinate. Next in step S410, the calculation processing unit 51 (pupil center calculator 56) acquires a pupil image and a pupil edge coordinate. The pupil image may be acquired by the sensor unit 23 or may be acquired by another sensor through image pickup. The image pickup may be performed by a sensor with a TOF function, and the pupil image may be acquired by using what is called a CMOS camera with no TOF function. Next in step S411, the calculation processing unit 51 (pupil center calculator 56) calculates a pupil center coordinate.

Next in step S412, the calculation processing unit 51 calculates the rotation angle of the eyeball based on the cornea center coordinate acquired in step S409 and the pupil center coordinate acquired in step S411. Next in step S413, the calculation processing unit 51 calculates a viewpoint coordinate and detects the line-of-sight direction of the user. Next in step S414, the calculation processing unit 51 stores data and performs communication with the control unit 61, the display unit 71, the memory 81, or the like.

### Fifth Embodiment

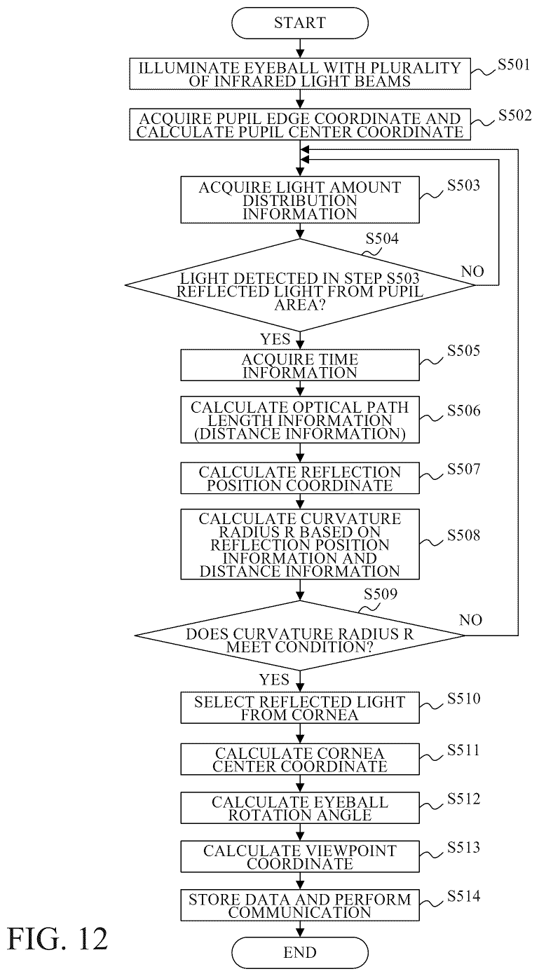

Next, operation of the measuring apparatus 1000b (measuring method) in a fifth embodiment of the present disclosure will be described below with reference to FIG. 12. FIG. 12 is a flowchart illustrating the operation of the measuring apparatus 1000b in the present embodiment. Each step in FIG. 12 is mainly executed by components of the measuring apparatus 1000b based on commands from the control unit 61. The basic configuration the measuring apparatus 1000b of the present embodiment is the same as that of the measuring apparatus 1000b of the fourth embodiment described above with reference to FIG. 10, and thus a description thereof will be omitted.

First in step S501, the illumination unit 11 illuminates an object such as a user's eye (eyeball) with light of a specific wavelength (infrared light), which is emitted from the light source unit 12 including a plurality of light sources. Next in step S502, the calculation processing unit 51 (pupil center calculator 56) acquires a pupil image from the photodetector 21 and calculates a pupil edge coordinate and a pupil center coordinate. Next in step S503, the photodetector 21 detects reflected light from the object and acquires light amount distribution information.

Next in step S504, the calculation processing unit 51 determines whether the reflected light detected in step S503 is reflected light from a pupil area based on the light amount distribution information. In a case where it is determined that the detected reflected light is not reflected light from a pupil area, the process returns to step S503 to acquire different light amount distribution information. In a case where it is determined that the detected reflected light is reflected light from a pupil area, the process proceeds to step S505. In step S505, the calculation processing unit 51 acquires the time of light emission from the light source unit 12 and the time of light reception at the sensor unit 23, and acquires time information from when light is emitted from the light source unit 12 to when the light reaches the sensor unit 23 after reflection by the object.

Next in step S506, the calculation processing unit 51 (distance calculator 521) calculates optical path length information (distance information) based on the light amount distribution information acquired in step S503 and the time information acquired in step S505. Next in step S507, the calculation processing unit 51 (imaging position calculator 522) calculates a reflective surface position coordinate (reflection position information) of the reflected light based on the optical path length information acquired in step S506. Next in step S508, the calculation processing unit 51 (reflective surface curvature radius calculator 523) calculates a radius R of curvature of the object (reflective surface) based on the reflection position coordinate (reflection position information) acquired in step S507 and the distance information acquired in step S506.

Next in step S509, the calculation processing unit 51 determines whether the radius R of curvature acquired in step S508 satisfies a predetermined condition, in other words, whether the radius R of curvature is within a predetermined range. In a case where it is determined that the radius R of curvature does not satisfy the predetermined condition, the process returns to step S503 to acquire different light amount distribution information. In a case where it is determined that the radius R of curvature satisfies the predetermined condition, the process proceeds to step 210.

In step S510, the calculation processing unit 51 (selection processing unit 55) selects reflected light for which the radius R of curvature is determined to be within the predetermined range as reflected light from a cornea. Next in step S511, the calculation processing unit 51 (corneal sphere center calculator 52b) selects a plurality of beams of reflected light from the cornea and calculates a cornea center coordinate.

Next in step S512, the calculation processing unit 51 calculates the rotation angle of the eyeball based on the cornea center coordinate acquired in step S511 and the pupil center coordinate acquired in step S501. Next in step S513, the calculation processing unit 51 calculates a viewpoint coordinate and detects the line-of-sight direction of the user. Next in step S514, the calculation processing unit 51 stores data and performs communication with the control unit 61, the display unit 71, the memory 81, or the like.

### Sixth Embodiment

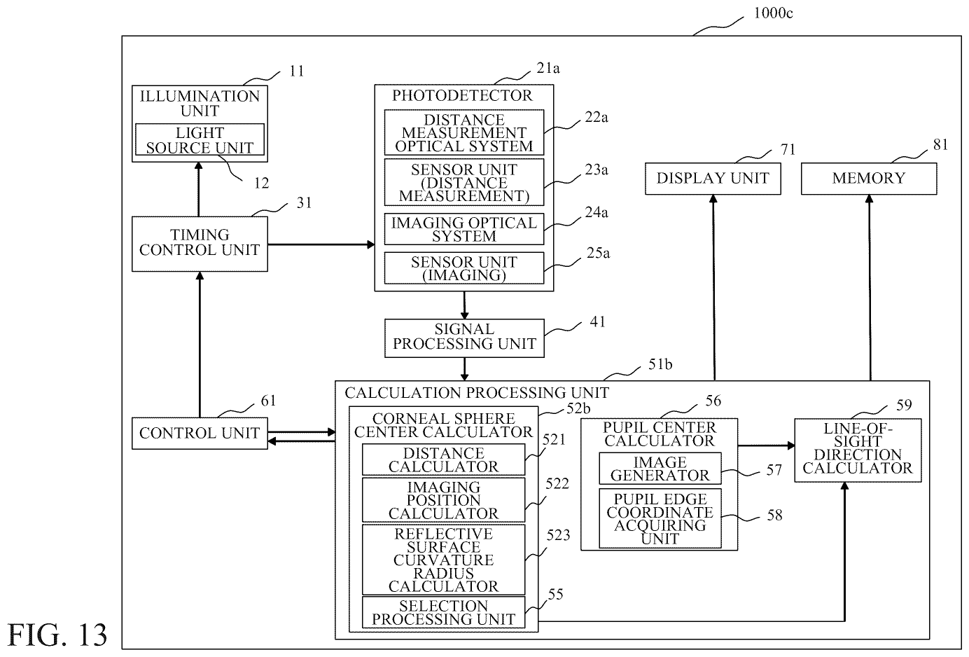

Next, a measuring apparatus 1000c in a sixth embodiment of the present disclosure will be described below with reference to FIG. 13. FIG. 13 is a block diagram of the measuring apparatus 1000c. The measuring apparatus 1000c is different from the measuring apparatus 1000b in that the photodetector 21a is provided in place of the photodetector 21. The other configuration of the measuring apparatus 1000c is the same as that of the measuring apparatus 1000b, and thus a description thereof will be omitted.

The photodetector 21a receives light (reflected light) reflected by an object. The photodetector 21a includes the distance-measuring optical system 22a, the sensor unit (distance measurement sensor unit) 23a, the imaging optical system 24a, and the sensor unit (image pickup sensor unit) 25a. The photodetector 21a condenses the reflected light through the distance-measuring optical system 22a to image the light onto the sensor unit 23a, and the photodetector 21a outputs a signal to the signal processing unit 41 in accordance with the light amount distribution information obtained by the sensor unit 23a, in other words, the intensity and acquisition time (time information) of the reflected light. In the present embodiment, the distance-measuring optical system 22a and the sensor unit 23a use a time of flight (TOF) method.

The imaging optical system 24a and the sensor unit 25a pick up an image of a user's eyeball (eye) to acquire an image for calculating a pupil center. The light amount distribution information obtained by the sensor unit 25a is output to the signal processing unit 41. The sensor unit 25a is an image sensor (photoelectric conversion element) such as a CCD sensor or a CMOS sensor.

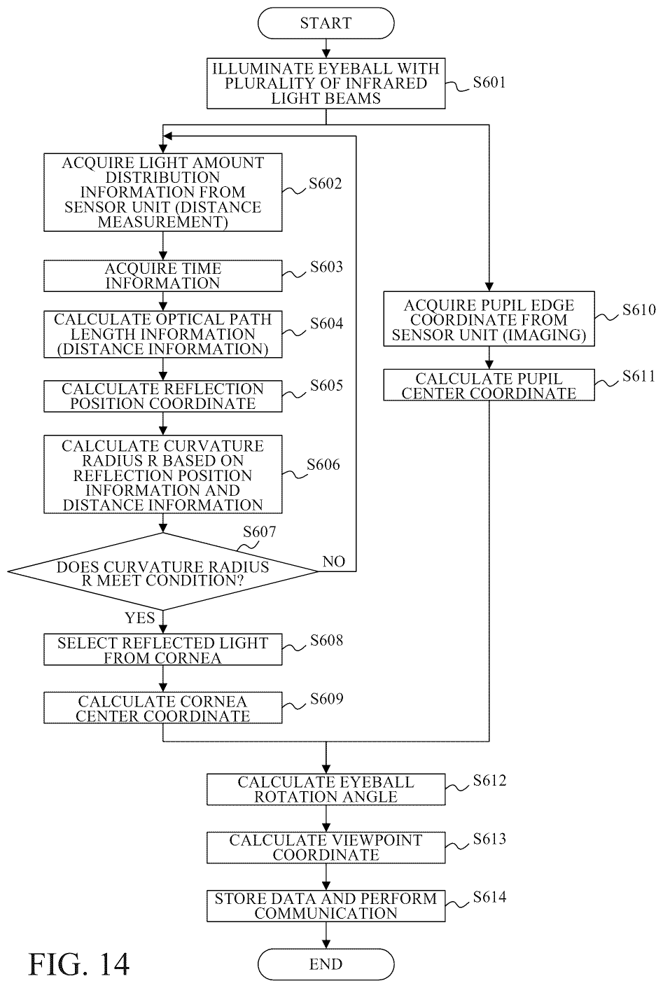

Next, the operation (measuring method) of the measuring apparatus 1000c in the present embodiment will be described below with reference to FIG. 14. FIG. 14 is a flowchart illustrating the operation of the measuring apparatus 1000c. Each step in FIG. 14 is mainly executed by components of the measuring apparatus 1000c based on commands from the control unit 61.

First in step S601, the illumination unit 11 illuminates an object such as a user's eye (eyeball) with light of a specific wavelength (infrared light), which is emitted from the light source unit 12 including a plurality of light sources. Next in step S602, the photodetector 21a detects reflected light from the object by using the sensor unit 23a and acquires light amount distribution information. Next in step S603, the calculation processing unit 51 acquires the time of light emission from the light source unit 12 and the time of light reception at the sensor unit 23a, and acquires time information from when light is emitted from the light source unit 12 to when the light reaches the sensor unit 23a after reflection by the object.

Next in step S604, the calculation processing unit 51 (distance calculator 521) calculates optical path length information (distance information) based on the light amount distribution information acquired in step S602 and the time information acquired in step S603. The optical path length information is information on an optical path length from when light is emitted from the light source unit 12 of the illumination unit 11 to when the light reaches the sensor unit 23a of the photodetector 21a after reflection by the object (reflective surface). Next in step S605, the calculation processing unit 51 (imaging position calculator 522) calculates a reflective surface position coordinate (reflection position information) of the reflected light based on the optical path length information acquired in step S604. Next in step S606, the calculation processing unit 51 (reflective surface curvature radius calculator 523) calculates a radius R of curvature of the object (reflective surface) based on the reflection position coordinate (reflection position information) acquired in step S605 and the distance information acquired in step S604.

Next in step S607, the calculation processing unit 51 determines whether the radius R of curvature acquired in step S606 satisfies a predetermined condition, in other words, whether the radius R of curvature is within a predetermined range. In a case where it is determined that the radius R of curvature does not satisfy the predetermined condition, the process returns to step S602 to acquire different light amount distribution information. In a case where it is determined that the radius R of curvature satisfies the predetermined condition, the process proceeds to step 308.

In step S608, the calculation processing unit 51 (selection processing unit 55) selects reflected light for which the radius R of curvature is determined to be within the predetermined range as reflected light from a cornea. Next in step S609, the calculation processing unit 51 (corneal sphere center calculator 52b) selects a plurality of beams of reflected light from the cornea and calculates a cornea center coordinate.

In step S610, the calculation processing unit 51 (pupil center calculator 56) acquires a pupil image from the sensor unit 25a and acquires a pupil edge coordinate. Next in step S611, the calculation processing unit 51 (pupil center calculator 56) calculates a pupil center coordinate.

Next in step S612, the calculation processing unit 51 calculates the rotation angle of the eyeball based on the cornea center coordinate acquired in step S609 and the pupil center coordinate acquired in step S611. Next in step S613, the calculation processing unit 51 calculates a viewpoint coordinate and detects the line-of-sight direction of the user. Next in step S614, the calculation processing unit 51 stores data and performs communication with the control unit 61, the display unit 71, the memory 81, or the like. The processing steps S602 to S609 and the processing steps S610 to S611 may be simultaneously performed.

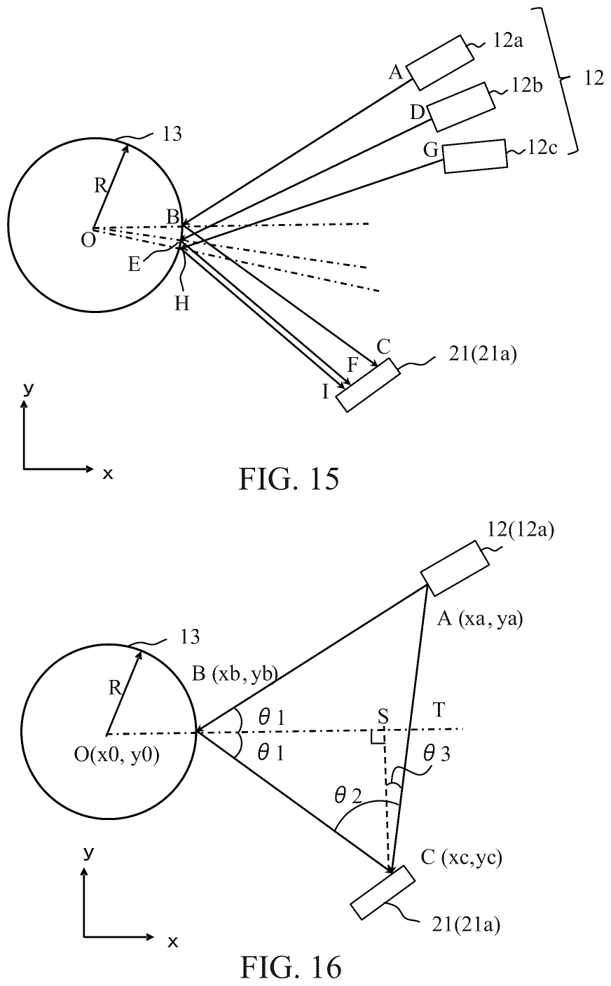

Next, examples of a method for calculating the reflective surface position coordinate and a method for calculating the radius R of curvature of the reflective surface will be described below with reference to FIGS. 15 and 16. FIG. 15 is an explanatory diagram of the measuring apparatus in each embodiment. FIG. 16 is an explanatory diagram of the method for calculating the radius R of curvature in each embodiment.

In FIG. 16, the light source position of the light source unit 12 is denoted by A(xa, ya), a position at which illumination light from the light source unit 12 (light source 12a) reaches an object and a light beam is reflected is denoted by a reflection position B(xb, yb), and the imaging position coordinate of the photodetector 21 (21a) is denoted by an imaging position C(xc, yc). The position coordinate of the corneal sphere center of eyeball an 13 is denoted by a corneal sphere center position O(x0, y0). The intersection point between the normal of a tangent line passing through the reflection position B of the object and a straight line AC connecting the light source position A and the imaging position C is denoted by T. A perpendicular line is drawn from the imaging position C to a straight line BT, and its intersection point is denoted by S. In addition, θ1=∠ABT=∠CBT, θ2=∠BCT, and θ3=∠BCS.

An optical path length from when a light beam is emitted from the light source position A to when the light beam reaches the imaging position C after reflection at the reflection position B is represented by L1, an optical path length from when a light beam is emitted from the light source position A to when the light beam is reflected by the reflection position B is represented by L11, and an optical path length from when a light beam is reflected at the reflection position B to when the light beam reaches the imaging position C is represented by L12. A linear distance connecting the light source position A and the imaging position C is represented by h. In this case, the following equation (1) holds.

$$L 1 = L 11 + L 12 \qquad ( 1 )$$

By applying the law of cosines to triangle ABC, the following equation (2) is obtained.

$$L 11 = \sqrt{L 1 {2}^{2} + {h}^{2} - 2 L 12 * h * cos Θ 2} \qquad ( 2 )$$

Since the linear distance h is known, it is possible to calculate the optical path lengths L11 and L12 from equations (1) and (2) by using information on the optical path length L1, which is acquired in each embodiment.

Then, by applying the law of sines to triangle BCS, equations (3) and (4) below are obtained.

$$\frac{L 12}{sin 90 °} = \frac{x c - x b}{sin ( Θ2 - Θ 3 )} \qquad ( 3 )$$

$$\frac{L 12}{sin 90 °} = \frac{y b - y c}{sin Θ1} \qquad ( 4 )$$

The light source position A(xa, ya) is known. In addition, the imaging position C(xc, yc), θ2, and θ3 are known from the relation between the sensor imaging position at which a light beam reaches the photodetector 21 (21a) and the angle of view. Thus, the reflection position B(xb, yb) can be calculated from equations (1) to (4).

Similarly, a reflection position E(xe, ye) of light from a light source 12b and a reflection position H(xh, yh) of light from a light source 12c illustrated in FIG. 15 can be calculated. Thus, the radius R of curvature of the reflective surface can be calculated by using the coordinates of the reflection positions B, E, and H, which are calculated based on the optical path lengths of light from the three light sources 12a to 12c, respectively, and the following equation (5).

$$R = \sqrt{{( x - x 0 )}^{2} + {( y - y 0 )}^{2}} \qquad ( 5 )$$

In each embodiment, the radius R of curvature (mm) may satisfy the following inequality (6).

$$6. < \vert R \vert < 10. \qquad ( 6 )$$

Inequality (6) defines the radius R of curvature (mm), in other words, the radius of curvature of the eyeball, which is calculated by the reflective surface curvature radius calculator 523. Inequality (6) is an inequality for determining whether the reflected light is reflected light from a cornea in step S407 or the like. By satisfying inequality (6), it is possible to more highly accurately determine whether the reflected light is from a cornea. In a case where the values becomes higher than the upper limit value of inequality (6), the radius of curvature becomes large, which makes it difficult to perform separation from the radius of curvature of glasses. In a case where the value becomes lower than the lower limit value of inequality (6), deviation from the corneal sphere radius increases.

The numerical range of inequality (6) may be set as in the following inequality (6a).

$$6.5 < R < 8.5 \qquad ( 6 a )$$

The numerical range of inequality (6a) may be set as in the following inequality (6b).

$$7. < R < 8. \qquad ( 6 b )$$

In each embodiment, light of a specific wavelength, which is emitted by the light source unit 12 of the illumination unit 11 may satisfy the following inequality (7).

$$600 < λ < 1500 \qquad ( 7 )$$

where λ (nm) is the central wavelength of the specific wavelength.

Inequality (7) defines the wavelength of light from the light source unit 12. By satisfying inequality (7), it is difficult for the user to perceive the light, and thus this is desirable.

The numerical range of inequality (7) may be set as in the following inequality (7a).

$$650 < λ < 1200 \qquad ( 7 a )$$

The numerical range of inequality (7a) may be set as in the following inequality (7b).

$$680 < λ < 900 \qquad ( 7 b )$$

The measuring apparatus of each embodiment does not necessarily need to be provided in a line-of-sight detection device. The measuring apparatus may be provided in, for example, a head-mounted display (HMD), or a movable unit such as an automobile. The measuring apparatus of each embodiment is also applicable to an ophthalmologic device such as a fundus camera or to an ophthalmologic examination system.

### Other Embodiments

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD™), a flash memory device, a memory card, and the like.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

Each embodiment can provide a measuring apparatus, a measuring method, and a storage medium, each of which can perform highly accurate line-of-sight detection.

What is claimed is:

1. A measuring apparatus for measuring a user's line-of-sight direction, the measuring apparatus comprising:
an illumination unit configured to emit light of a specific wavelength;
a photodetector having a plurality of two-dimensionally arranged photoelectric converters, and configured to acquire light amount distribution information on reflected light of the light emitted by the illumination unit;
one or more memories storing instructions; and
one or more processors that, upon execution of the instructions, operate to calculate the line-of-sight direction based on the light amount distribution information,
wherein the light amount distribution information includes information on a time taken from when the illumination unit emits the light to when the photodetector detects the reflected light or a distance corresponding to the time, and information on an intensity of the reflected light.

2. The measuring apparatus according to claim 1, wherein the one or more processors operate to:
store the light amount distribution information,
determine, based on the light amount distribution information, whether the reflected light is first reflected light from a cornea of the user or second reflected light that is unnecessary, and
calculate the line-of-sight direction based on the first reflected light.

3. The measuring apparatus according to claim 2, wherein the one or more processors operate to determine that the reflected light is the second reflected light in a case where the time or the distance is smaller than a first threshold value.

4. The measuring apparatus according to claim 2, wherein the one or more processors operate to determine that the reflected light is the second reflected light when the intensity is smaller than a second threshold value.

5. The measuring apparatus according to claim 1, wherein the illumination unit includes a plurality of light sources.

6. A measuring apparatus for measuring a user's line-of-sight direction, the measuring apparatus comprising:
an illumination unit configured to emit light of a specific wavelength;
a photodetector having a plurality of two-dimensionally arranged photoelectric converters, and configured to acquire light amount distribution information on reflected light of the light emitted by the illumination unit;
one or more memories storing instructions; and
one or more processors that, upon execution of the instructions, operate to:
calculate the line-of-sight direction based on the light amount distribution information,
calculate, based on the light amount distribution information, optical path length information from when the light is emitted by the illumination unit to when the light reaches the photodetector after reflection by a reflective surface,
calculate a radius of curvature of the reflective surface based on the optical path length information, and
calculate the line-of-sight direction based on the radius of curvature.

7. The measuring apparatus according to claim 6, wherein the illumination unit includes a plurality of light sources, and
wherein the one or more processors operate to:
calculate a plurality of pieces of optical path length information on the light emitted from the plurality of light sources, and
calculate the radius of curvature using the plurality of pieces of optical path length information.

8. The measuring apparatus according to claim 6, wherein the one or more processors operate to:
determine, based on the radius of curvature, whether the reflected light is first reflected light from a cornea of the user or second reflected light that is unnecessary, and
calculate the line-of-sight direction based on the first reflected light.

9. The measuring apparatus according to claim 8, wherein the second reflected light is reflected light from glasses of the user.

10. The measuring apparatus according to claim 8, wherein the one or more processors operate to determine that the reflected light is the first reflected light in a case where the radius of curvature is within a predetermined range.

11. The measuring apparatus according to claim 10, wherein the radius of curvature is determined to be within the predetermined range in a case where the following inequality is satisfied:
$$6. < \vert R \vert < 10.$$
where R (mm) is the radius of curvature.

12. The measuring apparatus according to claim 6, wherein the illumination unit includes three or more light sources.

13. The measuring apparatus according to claim 1, wherein the one or more processors operate to control an emission timing of the illumination unit and a detection timing of the photodetector.

14. The measuring apparatus according to claim 1, further comprising a counter configured to count light incident on each of the plurality of photoelectric converters.

15. The measuring apparatus according to claim 1, wherein the following inequality is satisfied:
$$600 < λ < 1500$$
where λ (nm) is a central wavelength of the specific wavelength.

16. The measuring apparatus according to claim 1, wherein the one or more processors operate to:
calculate a corneal sphere center position based on the light amount distribution information, and
calculate the line-of-sight direction based on the corneal sphere center position.

17. The measuring apparatus according to claim 1, wherein the one or more processors operate to:
calculate a pupil center position based on the light amount distribution information, and
calculate the line-of-sight direction based on the pupil center position.

18. The measuring apparatus according to claim 1, wherein the light emitted by the illumination unit is pulsed light.

19. The measuring apparatus according to claim 1, wherein each of the plurality of photoelectric converters is an avalanche diode.

20. The measuring apparatus according to claim 1, wherein an operation of emission start of the illumination unit and an operation of light detection start of the photodetector are each performed a plurality of times with a fixed duration from the emission start to the light detection start.

21. The measuring apparatus according to claim 1, wherein the measuring apparatus is provided in a HMD.

22. The measuring apparatus according to claim 1, wherein the measuring apparatus is provided in a fundus camera.

23. The measuring apparatus according to claim 1, wherein the measuring apparatus is provided in a movable unit.

24. A measuring method for measuring a user's line-of-sight direction, the measuring method comprising:
emitting light of a specific wavelength using an illumination unit;
acquiring light amount distribution information on reflected light of the light emitted by the illumination unit, using a photodetector having a plurality of two-dimensionally arranged photoelectric converters; and
calculating the line-of-sight direction based on the light amount distribution information,
wherein the light amount distribution information includes information on a time taken from when the illumination unit emits the light to when the photodetector detects the reflected light or a distance corresponding to the time, and information on an intensity of the reflected light.

25. A measuring method for measuring a user's line-of-sight direction, the measuring method comprising:
emitting light of a specific wavelength using an illumination unit;
acquiring light amount distribution information on reflected light of the light emitted by the illumination unit, using a photodetector having a plurality of two-dimensionally arranged photoelectric converters; and
calculating the line-of-sight direction based on the light amount distribution information,
wherein calculating the line-of-sight direction includes:
calculating, based on the light amount distribution information, optical path length information from when the light is emitted by the illumination unit to when the light reaches the photodetector after reflection by a reflective surface,
calculating a radius of curvature of the reflective surface based on the optical path length information, and
calculating the line-of-sight direction based on the radius of curvature.

26. A non-transitory computer-readable storage medium storing a program that causes a computer to execute the measuring method according to claim 24.

27. A non-transitory computer-readable storage medium storing a program that causes a computer to execute the measuring method according to claim 25.
